(19) United States
(12) Patent Application Publication
(10) Pub. No.: US 2025/0288253 A1
(43) Pub. Date: Sep. 18, 2025

(54) Proximity Detection With LDAF

(71) Applicant: Google LLC, Mountain View, CA (US)

(72) Inventors: Patrick Muller Amihood, Palo Alto, CA (US); Octavio Ponce Madrigal, Mountain View, CA (US); Sharanya Srinivas, San Jose, CA (US); Shandor Glenn Dektor, Sunnyvale, CA (US); Deborah Kathleen Vitus, San Mateo, CA (US)

(21) Appl. No.: 19/080,097

(22) Filed: Mar. 14, 2025

Related U.S. Application Data
(60) Provisional application No. 63/565,236, filed on Mar. 14, 2024.

(51) Int. Cl. A61B 5/00; A61B 5/01; G01S 17/04

(52) U.S. Cl. CPC A61B 5/6898; A61B 5/01; G01S 17/04; A61B 2562/0257

(57) ABSTRACT

Computing systems, computing devices, and computer-implemented methods are provided. In one aspect, a mobile computing device includes a display and an image capture assembly having an image capture device and one or more sensors. The mobile computing device further includes one or more processors configured to perform one or more operations. For instance, the operations may include obtaining optical sensor data, processing the optical sensor data to determine a proximity metric for the user, and, subsequent to determining the proximity metric for the user, determining one or more biometrics the user.

### PRIORITY CLAIM

The present application claims priority to U.S. Patent Application No. 63/565,236, titled “PROXIMITY DETECTION WITH LDAF,” having a filing date of Mar. 14, 2024, which is incorporated herein by reference.

### FIELD

The present disclosure relates generally to mobile computing devices.

### BACKGROUND

Computing devices capable of monitoring and detecting health-related information associated with a user of the computing device may track the user's activities and/or biometrics using a variety of sensors. Data captured from these sensors may be analyzed in order to provide the user with information such as, for instance, an estimation of their skin temperature, how far they walked in a day, their heart rate, how much time they spent sleeping, and the like. As additional capabilities are included in such computing devices, there is a need for improving the accuracy of, for example, skin temperature monitoring, using components that may already be present on the computing device.

### SUMMARY

Aspects and advantages of embodiments of the present disclosure will be set forth in part in the following description, or can be learned from the description, or can be learned through practice of the embodiments.

In an aspect, the present disclosure is directed to a method. The method includes obtaining, by a mobile computing device, optical sensor data via an optical sensor of the mobile computing device. The method further includes processing, by a ranging algorithm of the mobile computing device, the optical sensor data to determine a proximity metric for a user of the mobile computing device. The method further includes, subsequent to determining the proximity metric for the user, determining, by the mobile computing device, one or more biometrics of the user.

In some examples, obtaining the optical sensor data includes determining, by the mobile computing device, that the user is within a field-of-view (FOV) of a laser direct autofocus (LDAF) sensor of the mobile computing device; emitting, by an emitter of the LDAF sensor, one or more optical signals in a direction towards the user; and receiving, by a collector array of the LDAF sensor, one or more reflected optical signals associated with the one or more optical signals emitted by emitter, the collector array comprising a plurality of collectors.

In some examples, the collector array includes at least sixteen collectors.

In some examples, determining that the user is within the FOV of the LDAF sensor includes: obtaining, by a temperature sensor of the mobile computing device, temperature sensor data associated with an object in the FOV of the LDAF sensor; determining, by the mobile computing device, a temperature of the object in the FOV of the LDAF based on the temperature sensor data; determining, by the mobile computing device, that the temperature of the object in the FOV of the LDAF is within a threshold temperature range; and in response to determining that the temperature of the object in the FOV of the LDAF sensor is within the threshold temperature range, determining, by the mobile computing device, that the user is within the FOV of the LDAF sensor.

In some examples, the threshold temperature range includes temperatures in a range of about 30 degrees C. to about 40 degrees C.

In some examples, the optical sensor data includes a plurality of measurement frames, each measurement frame comprising a plurality of reflected optical signals respectively associated with a plurality of optical signals emitted by the optical sensor. In some examples, processing the optical sensor data to determine the proximity metric includes, for each measurement frame: determining, by the mobile computing device, a temporal relationship between the measurement frame and a preceding measurement frame of the plurality of measurement frames; determining, by the mobile computing device, that the measurement frame is temporally correlated with the preceding measurement frame based on the temporal relationship; in response to determining that the measurement frame is temporally correlated with the preceding measurement frame, determining, by the mobile computing device, a measurement region-of-interest (ROI) for the measurement frame, the measurement ROI comprising a subset of the plurality of reflected optical signals of the measurement frame; and storing, by the mobile computing device, the measurement ROI for each measurement frame in a memory of the mobile computing device.

In some examples, the subset is a first subset. In some examples, processing the optical sensor data to determine the proximity metric further includes, for each measurement frame: sorting, by the mobile computing device, the first subset of the plurality of reflected optical signals of the measurement ROI based on a magnitude of the respective reflected optical signal of the first subset; determining, by the mobile computing device, that one or more of the plurality of reflected optical signals in the measurement ROI are invalid optical signals based on the respective magnitude; discarding, by the mobile computing device, the invalid optical signals from the subset to generate a second subset comprising a plurality of valid reflected optical signals; and determining, by the mobile computing device, an average magnitude of the second subset based on the respective magnitude of each valid reflected optical signal of the plurality of valid reflected optical signals; and determining, by the mobile computing device, the proximity metric for the user based on the average magnitude of the second subset of each of the plurality of measurement frames.

In some examples, determining that one or more of the plurality of reflected optical signals in the measurement ROI are invalid optical signals includes, for at least one reflected optical signal in the measurement ROI: determining, by the mobile computing device, that a magnitude of the reflected optical signal is indicative of collector saturation; and in response to determining that the magnitude of the reflected optical signal is indicative of collector saturation, determining, by the mobile computing device, that the reflected optical signal is an invalid optical signal.

In some examples, determining that one or more of the plurality of reflected optical signals in the measurement ROI are invalid optical signals includes, for at least one reflected optical signal in the measurement ROI: determining, by the mobile computing device, that a collector of the optical sensor that received the reflected optical signal is within a threshold distance to an emitter of the optical sensor; and in response to determining that the collector of the optical sensor that received the reflected optical signal is within the threshold distance to the emitter, determining, by the mobile computing device, that the reflected optical signal is an invalid optical signal.

In some examples, the invalid optical signals include a reflected optical signal having a smallest magnitude of the plurality of reflected optical signals in the measurement ROI and a reflected optical signal having a greatest magnitude of the plurality of reflected optical signals in the measurement ROI.

In some examples, determining the one or more biometrics of the user includes: determining, by the mobile computing device, that the user is within a threshold testing distance from the mobile computing device based on the proximity metric; in response to determining that the user is within the threshold testing distance from the mobile computing device, generating, by the mobile computing device, a notification for display to the user via an output device of the mobile computing device, the notification indicating to the user that the user is within the threshold testing distance from the mobile computing device; obtaining, by the mobile computing device, biometric sensor data via one or more biometric sensors of the mobile computing device; and determining, by the mobile computing device, the one or more biometrics of the user based on the biometric sensor data.

In some examples, the threshold testing distance is less than about 30 millimeters.

In some examples, the notification is one of an auditory notification, a visual notification, or a haptic notification.

In some examples, the one or more biometrics of the user includes a body temperature of the user.

In some examples, the optical sensor is a laser direct autofocus (LDAF) sensor.

In some examples, the LDAF sensor comprises a sampling rate in a range of about 5 Hz to about 30 Hz.

In some examples, the proximity metric corresponds to a separation distance between the mobile computing device and a body part of the user.

In some examples, the separation distance is a distance between a forehead of the user and the optical sensor of the mobile computing device.

In another aspect, the present disclosure is directed to a mobile computing device. The mobile computing device includes a display, an image capture assembly comprising an imaging device, an optical sensor, a biometric sensor, and one or more processors. The one or more processors are configured to: obtain optical sensor data; process, with a ranging algorithm, the optical sensor data to determine a proximity metric for the user; and, subsequent to determining the proximity metric for the user, determine one or more biometrics of the user.

In another aspect, the present disclosure is directed to one or more non-transitory computer-readable media collectively storing instructions that, when executed by one or more processors of a mobile computing device, cause the mobile computing device to perform operations. The operations include: obtaining optical sensor data via an optical sensor of the mobile computing device; processing, by a ranging algorithm, the optical sensor data to determine a proximity metric for a user of the mobile computing device; and, subsequent to determining the proximity metric for the user, determining one or more biometrics of the user.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1 depicts a front view of an example mobile computing device according to example embodiments of the present disclosure;

FIG. 2 depicts a rear view of an example mobile computing device according to example embodiments of the present disclosure;

FIG. 3 depicts a side view of an example mobile computing device according to example embodiments of the present disclosure;

FIG. 4 depicts a block diagram of an example mobile computing device according to example embodiments of the present disclosure;

FIG. 5 depicts an example laser detect auto-focus (LDAF) sensor according to example embodiments of the present disclosure;

FIG. 6 depicts an example field of view (FOV) of an example LDAF sensor according to example embodiments of the present disclosure;

FIG. 7 depicts an example field of illumination (FOI) of an example LDAF sensor according to example embodiments of the present disclosure;

FIG. 8 depicts example zone mappings of an example LDAF sensor according to example embodiments of the present disclosure;

FIG. 9 depicts an example single-photon avalanche diode (SPAD) array of an example LDAF sensor according to example embodiments of the present disclosure;

FIG. 10 depicts a plan view of an example spatial arrangement of an example mobile computing device according to example embodiments of the present disclosure;

FIG. 11 depicts a side view of an example spatial arrangement of an example mobile computing device according to example embodiments of the present disclosure;

FIG. 12 depicts a flowchart diagram of an example ranging algorithm according to example embodiments of the present disclosure;

FIG. 13 depicts a block diagram of an example skin-gating framework according to example embodiments of the present disclosure; and

FIG. 14 depicts a flow chart diagram of an example method according to example embodiments of the present disclosure.

Repeat use of reference characters in the present specification and drawings is intended to represent the same and/or analogous features or elements of the present invention.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations may be made to the embodiments without departing from the scope or spirit of the present disclosure. For instance, features illustrated or described as part of one embodiment may be used with another embodiment to yield a still further embodiment. Thus, it is intended that aspects of the present disclosure cover such modifications and variations. Furthermore, it should be understood that the drawings are intended to represent structures for purposes of identification and description and are not intended to represent the structures to physical scale.

Recent consumer interest in personal health has led to a variety of personal health monitoring devices being offered in the market. These personal health monitoring devices have gained popularity amongst consumers due to their ability to monitor and determine a variety of health-related information associated with a user of the device. For example, devices such as fitness trackers, smartwatches, and/or the like are able to monitor and determine information relating to the pulse or motion of the user of the device.

Moreover, recent advances in sensor, electronics, and power source miniaturization have allowed the size of personal health monitoring devices (also referred to herein as “biometric tracking” or “biometric monitoring” devices) to be offered in extremely small sizes that were previously impractical. For example, certain biometric monitoring devices may include a variety of sensors for measuring multiple biological parameters that can be beneficial to a user of the device, such as a heart rate sensor, multi-purpose electrical sensors compatible with electrocardiogram (ECG) and electrodermal activity (EDA) applications, red and infrared sensors, an inertial measurement unit (IMU), a gyroscope, an altimeter, an accelerometer, a temperature sensor, an ambient light sensor, Wi-Fi, GPS, a vibration or haptic feedback sensor, a speaker, and a microphone, among others. However, the amount and types of health-related information capable of being monitored and determined by such devices has conventionally been limited.

Example aspects of the present disclosure are directed to a mobile computing device, such as a smartphone and/or the like, that is operable to determine one or more biometrics for a user, such as a body temperature of the user. More particularly, example aspects of the present disclosure are directed to a mobile computing device that is configured to determine a proximity metric for a user and, based on the proximity metric, determine a biometric for the user, such as a body temperature of the user. As described herein, a user device, such as the mobile computing device described herein, may include a laser detect auto-focus (LDAF) system that is operable to automatically focus one or more apertures/lenses of a image capture assembly (e.g., camera) for the mobile computing device. The mobile computing device may further include a biometric sensor, such as a temperature sensor. To ensure accurate biometric measurements, the mobile computing device utilizes the LDAF sensor to detect the user's proximity (e.g., proximity metric) to ensure the user is within a proper range for the temperature sensor.

As discussed in greater detail below, an emitter of a LDAF sensor emits signals and/or beams (e.g., laser pulses) towards a reflective portion of the user's body. In some examples, the LDAF sensor emits the laser pulses towards a forehead of the user, because the forehead is more reflective than other parts of the user's body. More particularly, typical human skin reflectance (e.g., forehead reflectance) may be in a range of about forty percent (40%) to about sixty percent (60%). This reduced reflectivity (e.g., versus other, more-reflective materials) is critical, because greater reflectivity may saturate the LDAF sensor (e.g., at the collector array) and, as a result, invalidate the proximity metric determination.

To ensure accurate biometric readings, the user must be within a threshold testing distance from the mobile computing device. In some examples, the threshold testing distance is about 15 millimeters. Due to various environmental factors, the LDAF sensor may not provide precise measurements below the threshold testing distance. However, example aspects of the present disclosure provide for accurate temperature measurements at any distance below the threshold testing distance. For instance, as an illustrative example, the user may be ten millimeters from the mobile computing device, but the LDAF sensor may determine that the user is approximately five millimeters from the mobile computing device. In such instances, however, the mobile computing device may nevertheless provide an accurate temperature biometric reading, because the user is within the threshold testing distance from the mobile computing device. Conversely, as another illustrative example, the user may be approximately twenty millimeters from the mobile computing device. In such instances, if the LDAF sensor determines that the user is within the threshold testing distance from the mobile computing device, the mobile computing device may provide an inaccurate temperature biometric reading due to the user being outside of the threshold testing distance.

Accordingly, an example LDAF sensor according to the present disclosure may include a plurality of collectors arranged in a grid-like pattern (e.g., an array). For instance, in some examples, the LDAF sensor may include at least sixteen collectors arranged in a four-by-four array, such as sixty-four collectors arranged in an eight-by-eight array. The LDAF sensor may receive the reflected signals at the collector array and may drop one or more reflected beams to reduce underestimation and/or overestimation, which may reduce the likelihood of inaccurate proximity metric determinations. Subsequently, the mobile computing device may calculate the average of the remaining reflected beams, which may then be used to calculate and determine the proximity metric for the user. In other words, an example LDAF sensor according to the present disclosure may carefully eliminate reflected beams that would otherwise provide an invalid and/or inaccurate proximity metric reading.

Once the proximity metric is determined, and the user is confirmed to be within the threshold distance, a notification may be surfaced to the user via, e.g., the display of the mobile computing device, which indicates that the mobile computing device is sufficiently proximate to the user's forehead, after which time the temperature sensor can be utilized to read a temperature near the user's forehead, temple, etc. In this manner, example aspects of the present disclosure ensure the temperature sensor of the mobile computing device is aligned with a target biometric-reading location, such as the user's forehead and/or the user's temple.

Example aspects of the present disclosure provide numerous technical effects and benefits. As an example, the systems and methods of the present disclosure provide improved techniques for obtaining and determining biometric information associated with a user. For instance, example aspects of the present disclosure provide for increased body-temperature readings by ensuring the user is within a threshold testing distance of the mobile computing device. Furthermore, by discarding reflected optical signals received by the collector array from the measurement region-of-interest (ROI), example aspects of the present disclosure are operable to minimize erroneous proximity determinations caused by overestimation and/or underestimation of the separation distance (e.g., between the user and the mobile computing device).

Furthermore, the systems and methods described herein may provide resulting improvements to computing technology tasked with monitoring and detecting biometric parameters in users. Improvements in the speed and accuracy of determining and detecting user biometric parameters can directly improve operational speeds for computing systems. For instance, by improving diagnostic accuracy (e.g., by reducing erroneous proximity determinations caused by invalid reflected optical signals), the number of duplicative diagnostic operations can be reduced-thereby reducing processing and storage requirements for the computing systems. Hence, the reduced processing and storage requirements ultimately result in more efficient resource use for the computing system. In this way, valuable computing resources within a computing system that would have otherwise been needed for such tasks may be reserved for other tasks.

As used herein, the terms “first,” “second,” and “third” may be used interchangeably to distinguish one component from another and are not intended to signify location or importance of the individual components. The terms “includes” and “including” are intended to be inclusive in a manner similar to the term “comprising.” Similarly, the term “or” is generally intended to be inclusive (e.g., “A or B” is intended to mean “A or B or both”). The term “at least one of” in the context of, e.g., “at least one of A, B, and C” refers to only A, only B, only C, or any combination of A, B, and C. In addition, here and throughout the specification and claims, range limitations may be combined and/or interchanged. Such ranges are identified and include all the sub-ranges contained therein unless context or language indicates otherwise. For example, all ranges disclosed herein are inclusive of the endpoints, and the endpoints are independently combinable with each other. The singular forms “a,” “an,” and “the” include plural references unless the context clearly dictates otherwise.

Approximating language, as used herein throughout the specification and claims, may be applied to modify any quantitative representation that could permissibly vary without resulting in a change in the basic function to which it is related. Accordingly, a value modified by a term or terms, such as “generally,” “about,” “approximately,” and “substantially,” are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a 10 percent margin, i.e., including values within ten percent greater or less than the stated value. In this regard, for example, when used in the context of an angle or direction, such terms include within ten degrees greater or less than the stated angle or direction, e.g., “generally vertical” includes forming an angle of up to ten degrees in any direction, e.g., clockwise or counterclockwise, with the vertical direction V.

The word “exemplary” is used herein to mean “serving as an example, instance, or illustration.” In addition, references to “an embodiment” or “one embodiment” do not necessarily refer to the same embodiment, although it may. Any implementation described herein as “exemplary” or “an embodiment” is not necessarily to be construed as preferred or advantageous over other implementations. Moreover, each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

Relative terms such as “below” or “above” or “upper” or “lower” or “horizontal” or “lateral” or “vertical” may be used herein to describe a relationship of one element, layer or region to another element, layer or region as illustrated in the figures. It will be understood that these terms are intended to encompass different orientations of the device in addition to the orientation depicted in the figures. Furthermore, unless otherwise defined, all terms (including technical and scientific terms) used herein have the same meaning as commonly understood by one of ordinary skill in the art to which this invention belongs. It will be further understood that terms used herein should be interpreted as having a meaning that is consistent with their meaning in the context of this specification and the relevant art and will not be interpreted in an idealized or overly formal sense unless expressly so defined herein.

In the drawings and specification, there have been disclosed typical embodiments and, although specific terms are employed, they are used in a generic and descriptive sense only and not for purposes of limitation of the scope set forth in the following claims. Furthermore, like numbers refer to like elements throughout. Thus, the same or similar numbers may be described with reference to other drawings even if they are neither mentioned nor described in the corresponding drawing. Also, elements that are not denoted by reference numbers may be described with reference to other drawings.

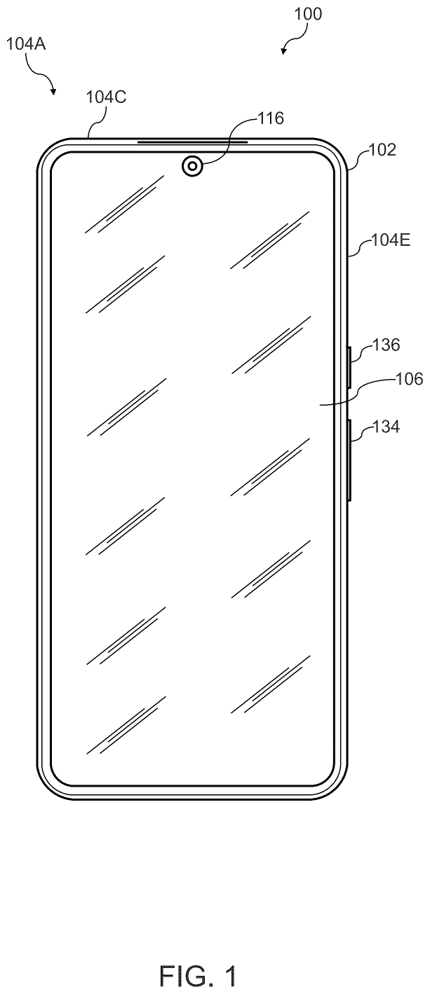

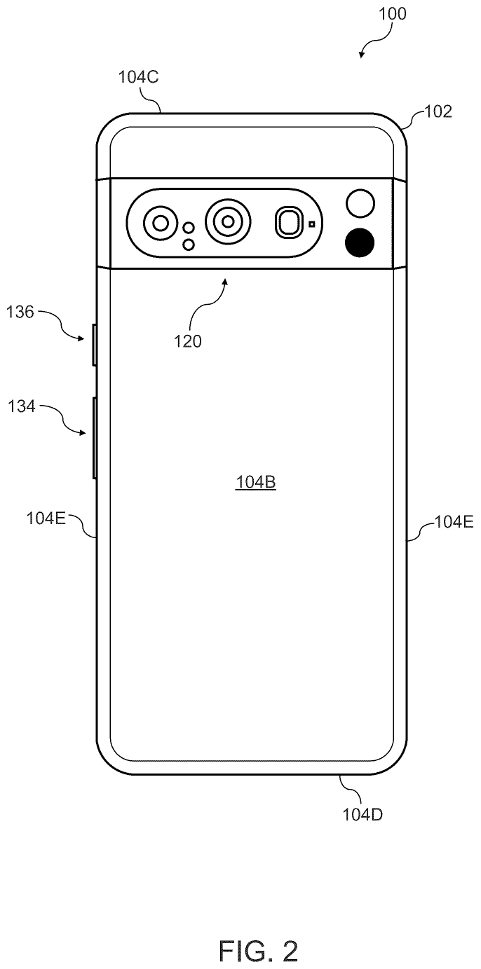

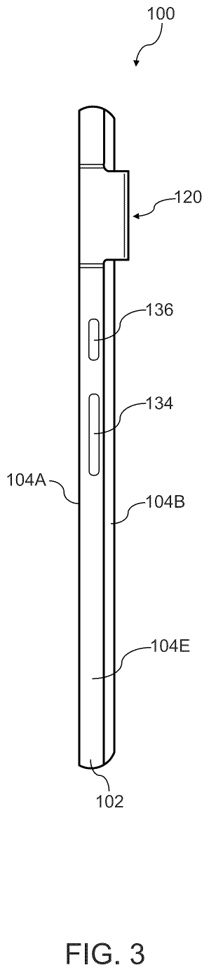

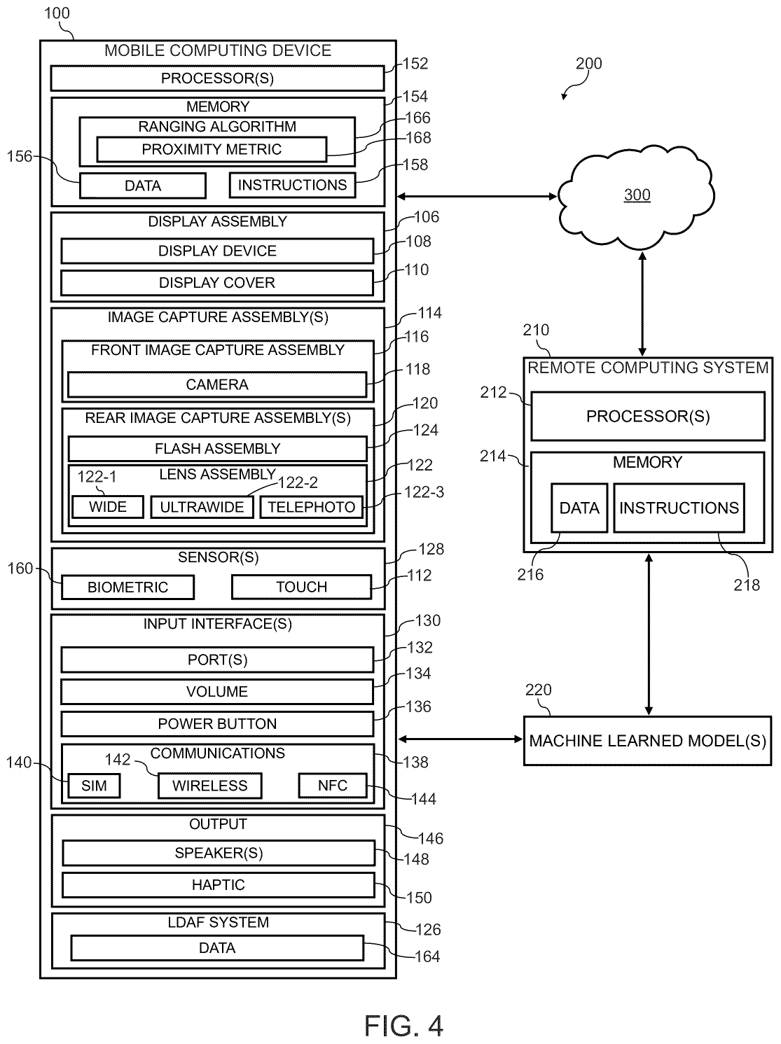

FIGS. 1-4 depict an example mobile computing device 100 according to example embodiments of the present disclosure. More particularly, FIG. 1 depicts a front view of the example mobile computing device 100. FIG. 2 depicts a rear view of the example mobile computing device 100. FIG. 3 depicts a side view of the example mobile computing device 100. FIG. 4 depicts a block diagram of example components of the example mobile computing device 100. It should be understood that FIGS. 1-4 depict the example mobile computing device 100 and its various components for purposes of illustration and discussion. Those having ordinary skill in the art, using the disclosures provided herein, will appreciate that example aspects of the present disclosure may be implemented by any suitable computing device, such as, by way of non-limiting example, a mobile tablet device, a wearable computing device, and the like.

Referring now to FIGS. 1-4, the mobile computing device 100 may include a housing 102. The housing 102 may include any suitable material, such as aluminum, titanium, and the like. As will be discussed in greater detail below, the housing 102 may define a front surface 104A (e.g., front side), a back surface 104B (e.g., back side), a top surface 104C (e.g., top side), a bottom surface 104D (e.g., bottom side), and one or more side surfaces 104E (e.g., left side, right side, etc.) of the mobile computing device. As shown, the front surface 104A and the back surface 104B of the mobile computing device 100 may be major surfaces/sides, while the top surface 104C, the bottom surface 104D, and the side surface(s) 104E may be minor surfaces/sides. The housing 102 may further define a cavity (e.g., internal volume) (not shown) in which one or more electronic components (e.g., disposed on printed circuit boards) are disposed. For instance, the mobile computing device 100 may include a printed circuit board (e.g., flexible printed circuit board) (not shown) disposed within the cavity defined by the housing 102. The mobile computing device 100 may further include a battery (not shown) that is disposed within the cavity defined by the housing 102. Furthermore, the mobile computing device 100 may also include one or more internal temperature sensors (not shown) within the cavity defined by the housing 102 that are configured to obtain internal temperature data indicative of an internal temperature of the mobile computing device 100.

The mobile computing device 100 may include a display assembly 106. The display assembly 106 may define the front surface 104A (e.g., front side) of the mobile computing device 100. The display assembly 106 may include a display device 108, which may be configured to display content (e.g., time, date, biometric, notifications, etc.) for viewing by the user and to receive inputs from the user. Furthermore, as discussed below, the display assembly 106 may include and/or otherwise be coupled to one or more touch sensors (e.g., touch sensors 112). Hence, the display assembly 106 may be a touch-sensitive display assembly 106 that is sensitive to the touch of a user object (e.g., finger, stylus, and the like). The touch-sensitive display assembly 106 may, in some examples, serve to implement, for instance, a virtual keyboard.

More particularly, in some examples, the display assembly 106 may include the display device 108. The display device 108 may include a plurality of pixels. For instance, in some examples, the display device 108 may include an organic light-emitting diode (OLED) display. It should be understood, however, that the display device 108 may include any suitable display device 108 without deviating from the scope of the present disclosure. In some examples, the display device 108 may be an “always-on” display operable to display content to the user in a quickly accessible way (e.g., “At a Glance”). In particular, in some examples, content may be displayed on the display device 108 even when the user is not explicitly interacting with the mobile computing device 100. In this manner, users may quickly access information by viewing content and performing actions without needing to invoke the computing system (e.g., performing “wake up” functions to activate the computing system).

The display assembly 106 may further include a display cover 110 positioned on the housing 102 such that the display cover 110 is positioned on top of the display device 108. In this manner, the display cover 110 may protect the display device 108 from being damaged (e.g., scratched or cracked). In some examples, the display assembly 106 may include a seal positioned between the housing 102 and the display cover 110. For instance, a first surface of the seal may contact the housing 102 and a second surface of the seal may contact the display cover 110. In this manner, the seal between the housing 102 and the display cover 110 may prevent a liquid (e.g., water) from entering the cavity defined by the housing 102. It should be understood that the display cover 110 may be optically transparent so that the user may view information being displayed on the display device 108. For instance, in some examples, the display cover 110 may include a glass material. It should be understood, however, that the display cover 110 may include any suitable optically transparent material.

As noted above, the display assembly 106 may further include and/or otherwise be coupled to one or more touch sensors 112 operable to detect one or more inputs (e.g., touch inputs) provided by the user, such as when the user touches and/or otherwise makes contact with the display assembly 106 (e.g., display cover 110). In this manner, the display assembly 106 may be a touch-sensitive display assembly 106. In some examples, one or more of the touch sensors 112 may include a capacitive sensor whose capacitance changes when a touch input is provided at a location on the display cover 110 that corresponds to the capacitive sensor. It should be understood, however, that the touch sensors 112 may include any suitable type of sensor configured to detect a touch input provided by the user touching the display assembly 106 (e.g., display cover 110).

The mobile computing device 100 may include one or more image capture assemblies 114. For instance, the mobile computing device 100 may include a front image capture assembly 116 on/within the front surface 104A of the mobile computing device 100. The front image capture assembly 116 may include, for instance, a front-facing camera 118 operable to capture images and/or videos. In some examples, the front image capture assembly 116 may be operable to implement a variety of image capture-related tasks, such as autofocus of an aperture and/or lens and the like. It should be understood that the front image capture assembly 116 may include any suitable image capture device without deviating from the scope of the present disclosure.

The mobile computing device 100 may further include a rear image capture assembly 120 on a rear/back surface 104B of the mobile computing device 100. In some examples, such as that depicted in FIGS. 1-4, the rear image capture assembly 120 may include a plurality of image capture devices (e.g., lens assembly 122) operable to capture images and/or videos. For instance, in some examples, the rear image capture assembly 120 may include a wide camera 122A, an ultrawide camera 122B, and a telephoto camera 122C. The rear image capture assembly 120 may also include a flash device (e.g., flash assembly 124), such as an LED flash. In some examples, the rear image capture assembly 120 may be operable to implement a variety of image capture-related tasks, such as auto focus of an aperture and/or lens (e.g., lens assembly 122), lens correction, zoom, optical and/or electronic image stabilization, and/or the like. By way of non-limiting example, as described below, the rear image capture assembly 120 may include and/or otherwise be coupled to a laser detect auto-focus (LDAF) system 126 operable to automatically focus one or more apertures/lenses (e.g., lens assembly 122) for the mobile computing device 100.

As will be discussed in greater detail below, the mobile computing device 100 may include (and receive data from) one or more sensors 128. The sensor(s) 128 may be housed in the housing 102. The sensor(s) 128 may include one or more image sensors (e.g., image capture devices discussed above), one or more LIDAR sensors, one or more audio sensors (e.g., microphone(s)), one or more inertial sensors (e.g., inertial measurement unit(s) (IMU(s))), one or more biometric sensors (e.g., heart rate sensor(s), pulse sensor(s), retinal sensor(s), fingerprint sensor(s), etc.), one or more touch sensors (e.g., conductive touch sensor(s), mechanical touch sensor(s)) (discussed above), one or more infrared (IR) sensors, one or more optical sensors, one or more location sensors (e.g., GPS), one or more temperature sensors, and/or one or more other sensors. The sensor(s) 128 may be used to obtain data associated with the user's environment (e.g., an image of a user's environment, a recording of the environment, a location of the user, an authentication of the user, a temperature of the user and/or an object in the environment, etc.). It should be understood that the sensor(s) 128 may include any suitable sensor without deviating from the scope of the present disclosure. For instance, as described in greater detail below, the mobile computing device 100 may include one or more biometric sensors 160 operable to obtain biometric sensor data, such as one or more temperature sensors operable to obtain body temperature data associated with a user of the mobile computing device 100.

The mobile computing device 100 may further include one or more buttons and/or ports (hereinafter, input interface elements 130). For instance, in some examples, the mobile computing device 100 may include a power port 132 operable to provide charge to the battery. The mobile computing device 100 may also include one or more volume buttons 134 operable to control a volume of audio output by one or more speakers. The mobile computing device 100 may also include a power button 136 operable to control a power state (e.g., “ON,” “OFF,” “IDLE,” “STANDBY,” etc.) of the mobile computing device 100. It should be understood that the mobile computing device 100 may include any suitable button and/or port without deviating from the scope of the present disclosure.

As discussed in greater detail below, the mobile computing device 100 may include one or more communications interfaces 138, which may be operable to communicate with remote computing systems and devices and/or third-party computing systems and devices over a variety of telecommunications networks. For instance, the mobile computing device 100 may include a Subscriber Identity Module (SIM) card 140, which, in conjunction with one or more antennas (e.g., mmWave antenna), allows the mobile computing device 100 to communicate over one or more telecommunications networks, such as a cellular network and the like. The mobile computing device 100 may also include a wireless communication interface 142, which may be operable to connect to wireless networks, such as local area networks, Wi-Fi networks, and the like. Even further, the mobile computing device 100 may also include a Near Field Communication (NFC) interface 144, which may include components operable to provide NFC capabilities to the mobile computing device 100.

In some examples, the mobile computing device 100 may include one or more output devices 146. For instance, as noted above, the output device(s) 146 may include the display device 108. The output device(s) 146 may further include one or more speakers 148. In this manner, the mobile computing device 100 may emit audible noises (e.g., alarm, voice automated messages, audio, etc.) for the user. The output device(s) 146 may further include one or more haptic devices 150 operable to provide one or more haptic notifications (e.g., vibratory notifications) to the user. It should be appreciated that the mobile computing device 100 may include any suitable output device without deviating from the scope of the present disclosure.

The mobile computing device 100 may further include one or more processors 152 and a memory 154. The processor(s) 152 may include any suitable processing device (e.g., a processor core, a microprocessor, an application specific integrated circuit (AISC), a field programmable gate array (FPGA), a microcontroller, etc.). In some examples, the processor(s) 152 may be communicatively coupled to the sensor(s) 128. For instance, the processor(s) 152 may be communicatively coupled to the sensor(s) 128 via a data interface (e.g., data bus). In this manner, the processor(s) 152 may obtain data from the sensor(s) 128. In some examples, the processor(s) 152 may determine one or more metrics, such as a proximity metric, a biometric, and/or the like, based on the data obtained from the sensor(s) 128. The memory 154 may include one or more non-transitory computer-readable storage media, such as random-access memory (RAM), read-only memory (ROM), electronically erasable programmable ready-only memory (EEPROM), erasable programmable read-only memory (EPROM), flash memory devices, and combinations thereof. The memory may store data 156 and instructions 158 that, when executed by the processor(s) 152, cause the processor(s) 152 to perform one or more operations, such as any of the operations disclosed herein.

As described in greater detail below, the mobile computing device 100 may be configured to implement a proximity determination framework (e.g., via the LDAF system 126) to ensure biometric sensor data 162 obtained by the biometric sensor(s) 160 of the mobile computing device 100 is accurate. More particularly, the mobile computing device 100 may be configured to obtain optical sensor data 164 via an optical sensor, such as an LDAF sensor of the LDAF system 126. The mobile computing device 100 may be further configured to process the optical sensor data 164 with a ranging algorithm 166 to determine a proximity metric 168 for a user of the mobile computing device 100. Subsequently, the mobile computing device 100 may determine one or more biometrics for the user of the mobile computing device 100, such as a body temperature of the user, based on the biometric sensor data 162 obtained by the biometric sensor(s) 160.

As noted above, the mobile computing device 100 may be part of a computing system 200, which may be operable to implement any of the methods and/or operations disclosed herein. The computing system 200 may include the mobile computing device 100 and a remote computing system 210. The mobile computing device 100 may be communicatively coupled to the remote computing system 210 over a network 300. As noted above, the network 300 may be any type of communications network and/or telecommunications network, such as, by way of non-limiting example, a local area network (e.g., intranet), wide area network (e.g., Internet), and/or some combination thereof. Furthermore, the network 300 may include any number of wired or wireless links. In general, communication over the network 300 may be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The remote computing system 210 may include one or more processors 212 and a memory 214. The processor(s) 212 may be any suitable processing device (e.g., a processor core, a microprocessor, an ASIC, an FPGA, a controller, a microcontroller, etc.) and may be one processor or a plurality of processors that are operatively connected. The memory 214 may include one or more non-transitory computer-readable storage medium(s), such as RAM, ROM, EEPROM, EPROM, flash memory devices, magnetic disks, etc., and combinations thereof. The memory 214 may store data 216 and instructions 218 which are executed by the processor 212 to cause the remote computing system 210 to perform operations, such as any of the operations described herein. In this manner, the remote computing system 210 may be operable to implement any of the methods described herein.

In some examples, the remote computing system 210 may include or may otherwise be implemented by one or more computing devices. In instances in which the remote computing system 210 includes plural server computing devices, such server computing devices may operate according to sequential computing architectures, parallel computing architectures, or some combination thereof.

Furthermore, the computing system 200 may include one or more machine-learned models 220. For instance, the machine-learned models 220 may be or may otherwise include various machine-learned models such as neural networks (e.g., deep neural networks) or other types of machine-learned models, including non-linear models and/or linear models. Neural networks may include feed-forward neural networks, recurrent neural networks (e.g., long short-term memory recurrent neural networks), convolutional neural networks or other forms of neural networks.

In some examples, the machine-learned model(s) 220 may be received from the remote computing system 210 over the network 300 and stored in the memory 154 of the mobile computing device 100. In such examples, the machine-learned model(s) 220 may then be used and/or otherwise implemented by the mobile computing device 100. In some examples, the mobile computing device 100 may implement multiple parallel instances of a single machine-learned model (e.g., to perform parallel machine-learned model processing across multiple instances of input data and/or detected features).

More particularly, the machine-learned model(s) 220 may include one or more detection models, one or more classification models, one or more segmentation models, one or more augmentation models, one or more generative models, one or more natural language processing models, one or more optical character recognition models, and/or one or more other machine-learned models. The machine-learned model(s) 220 may include one or more transformer models. The machine-learned model(s) 220 may include one or more neural radiance field models, one or more diffusion models, and/or one or more autoregressive language models.

The machine-learned model(s) 220 may be utilized to detect one or more object features. The detected object features may be classified and/or embedded. The classification and/or the embedding may then be utilized to perform a search to determine one or more search results. Alternatively and/or additionally, the one or more detected features may be utilized to determine an indicator (e.g., a user interface element that indicates a detected feature) is to be provided to indicate a feature has been detected. The user may then select the indicator to cause a feature classification, embedding, and/or search to be performed. In some implementations, the classification, the embedding, and/or the searching may be performed before the indicator is selected.

In some examples, the machine-learned model(s) 220 may process image data, text data, audio data, and/or latent encoding data to generate output data that may include image data, text data, audio data, and/or latent encoding data. The machine-learned model(s) 220 may perform optical character recognition, natural language processing, image classification, object classification, text classification, audio classification, context determination, action prediction, image correction, image augmentation, text augmentation, sentiment analysis, object detection, error detection, inpainting, video stabilization, audio correction, audio augmentation, and/or data segmentation (e.g., mask based segmentation).

Additionally and/or alternatively, machine-learned model(s) 220 may be included in or otherwise stored and implemented by the server computing system (e.g., remote computing system 210) that communicates with the mobile computing device 100 according to a client-server relationship. For instance, the machine-learned model(s) 220 may be implemented by the remote computing system 210 as a portion of a web service (e.g., a viewfinder service, a visual search service, an image processing service, an ambient computing service, and/or an overlay application service). Thus, one or more models may be stored and implemented at the mobile computing device 100 and/or one or more models may be stored and implemented at the remote computing system 210.

The technology discussed herein refers to sensors and other computer-based systems, as well as actions taken, and information sent to and from such systems. One of ordinary skill in the art will recognize that the inherent flexibility of computer-based systems allows for a great variety of possible configurations, combinations, and divisions of tasks and functionality between and among components. For instance, server processes discussed herein may be implemented using a single server or multiple servers working in combination. Databases and applications may be implemented on a single system or distributed across multiple systems. Distributed components may operate sequentially or in parallel.

FIGS. 1-4 and the corresponding discussion illustrate and describe an example mobile communication device 100 for purposes of illustration and discussion. In particular, FIGS. 1-4 depict a Google Pixel™ 8 Pro, the technical specifications (URL: https://support.google.com/pixelphone/answer/7158570?hl=en&ref_topic=7530176&sjid=32349 2628379524691-NA) of which are incorporated herein by reference. It should be understood, however, that any suitable mobile computing device 100 may be used without deviating from the scope of the present disclosure.

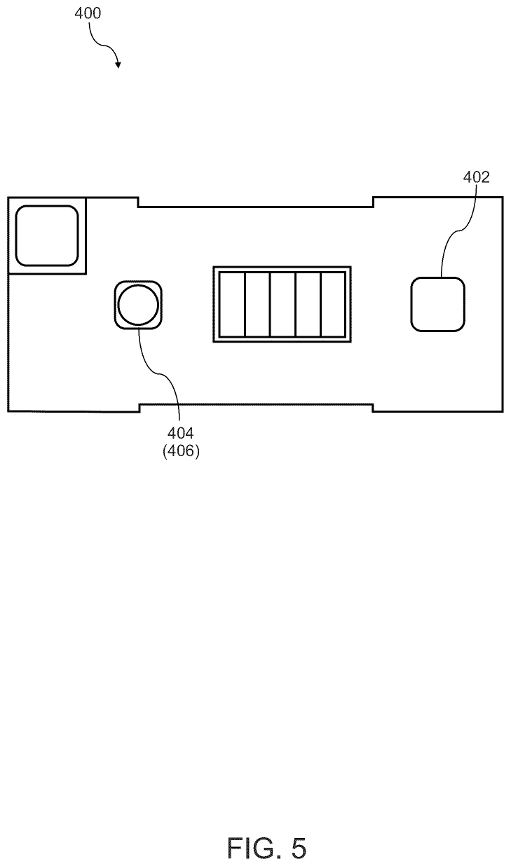

As noted above, the rear image capture assembly 120 of the example mobile computing device 100 discussed herein may include and/or otherwise be coupled to a laser detect auto-focus (LDAF) system 126 that provides autofocus capabilities to the rear image capture assembly 120. By way of non-limiting example, an example LDAF sensor system 126 will now be discussed with reference to FIGS. 5-9. FIGS. 5-9 will be discussed in conjunction with FIGS. 1-4.

FIG. 5 depicts an example LDAF sensor 400 according to example embodiments of the present disclosure. As noted above, the LDAF sensor 400 may be part and/or otherwise be coupled to the LDAF system 126. The LDAF sensor 400 may be an optical sensor, such as a laser-based optical sensor. The LDAF sensor 400 may be part of the rear image capture assembly 120 (FIGS. 1-4) and may be configured to assist in focusing the lenses of the lens assembly 122 (FIGS. 1-4) and/or apertures of the image capture device(s) by determining one or more proximity metrics (e.g., proximity metric 168) for a subject and/or target. It should be understood that the terms “subject” and “target” may be used interchangeably. For instance, LDAF sensors 400 according to the present disclosure are operable to estimate distances in millimeter precision. For instance, by way of non-limiting example, an LDAF sensor 400 according to the present disclosure may be operable to estimate distances between about 1 millimeter to about 400 centimeters.

More particularly, the LDAF sensor 400 may include at least one emitter 402 (e.g., transmitter) and a plurality of receivers and/or collectors (hereinafter, collectors 404). It should be understood that, as used herein, the terms “receivers” and “collectors” may be used interchangeably. The at least one emitter 402 of the LDAF sensor 400 may transmit laser pulses, such as pulses having a wavelength in a range of about 900 nanometers to about 1000 nanometers, such as about 940 nanometers. The plurality of collectors 404 may be an array of collectors 404, such as an array of single-photon avalanche diode (SPAD) imagers (hereinafter “SPAD array” and/or “collector array” and/or “array”) arranged in a grid-like pattern. For instance, in some examples, the LDAF sensor 400 may include a collector array 406 having at least four collectors 404 arranged in a two-by-two grid. In some examples, the LDAF sensor 400 may include a collector array 406 having at least nine collectors 404 arranged in a three-by-three grid. In some examples, the LDAF sensor 400 may include a collector array 406 having at least sixteen collectors 404 arranged in a four-by-four grid. In some examples, the LDAF sensor 400 may include a collector array 406 having at least sixty-four collectors 404 arranged in an eight-by-eight grid. In some examples, the LDAF sensor 400 may include a collector array 406 having at least 144 collectors 404 arranged in a twelve-by-twelve array. In some examples, the LDAF sensor 400 may include a collector array 406 having at least 256 collectors 404 arranged in a sixteen-by-sixteen array. In some examples, the LDAF sensor 400 may include a collector array 406 having at least 400 collectors 404 arranged in a twenty-by-twenty array. It should be understood, however, that the collector array 406 may include any suitable number of collectors 404 arranged in any suitable array having any suitable dimensions without deviating from the scope of the present disclosure.

The emitter 402 may transmit a plurality of optical signals (e.g., laser pulses) towards a subject. Depending on a variety of environmental and hardware-related factors, one or more of the optical signals may be reflected back towards the LDAF sensor 400. Subsequently, the plurality of collectors 404 may receive the scattered energy (e.g., reflected laser pulses) as echoes (hereinafter, “reflected” optical signals). The LDAF sensor 400 may be configured to measure, inter alia, the time it takes for the laser pulses emitted from the at least one emitter 402 to be received by the plurality of collectors 404. Based on this timing, the mobile computing device 100 (FIGS. 1-4) may calculate and determine a distance between the LDAF sensor 400 and the subject.

In some examples, the LDAF sensor 400 may have a sample rate, for instance, in a range of about 5 Hz to about 50 Hz, such as a range of about 5 Hz to about 30 Hz, such as a range of about 10 Hz to about 25 Hz, such as a range of about 12.5 Hz to about 20 Hz, such as about 15 Hz.

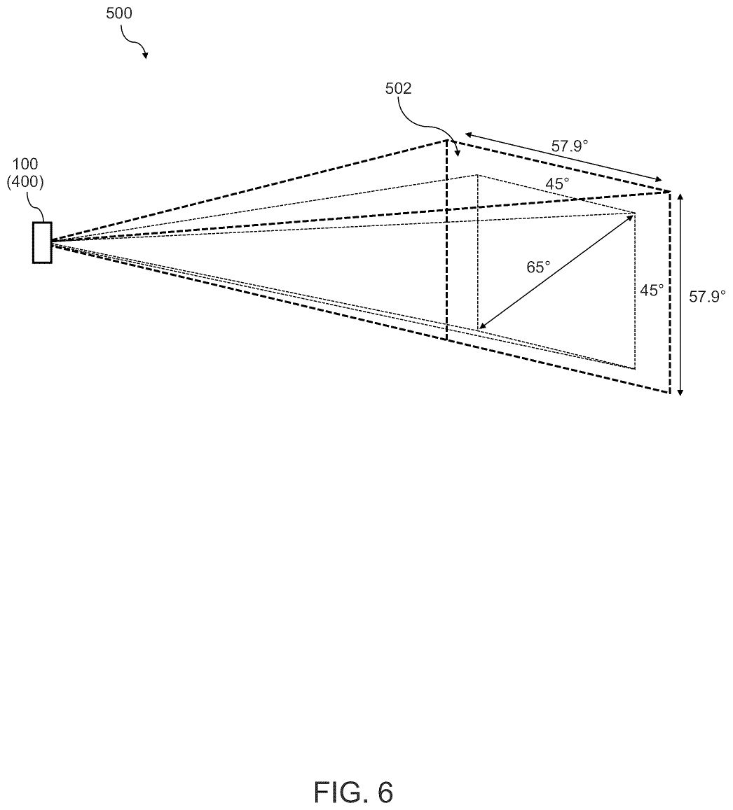

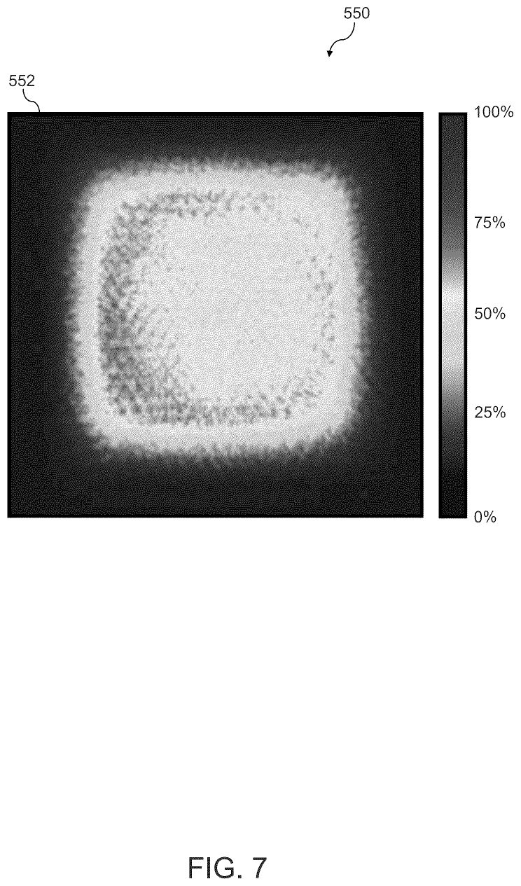

FIGS. 6-7 depict an example field of view (FOV) 500 and an example field of illumination (FOI) 550, respectively, of the example LDAF sensor 400 according to example embodiments of the present disclosure. As used herein, the terms “field of view” or “FOV” and “field of illumination” or “FOI” are used to describe the area over which the LDAF sensor 400 may effectively operate. More particularly, “field of view” or “FOV” refers to the angle and/or area within which the LDAF sensor 400 may detect a particular subject. For instance, a “wider” FOV 500 means the LDAF sensor 400 may be operable over a larger area, while a “narrower” FOV 500 means the LDAF sensor 400 may be operable over a smaller area. In some examples, the LDAF sensor 400 according to the present disclosure may have an FOV 500 in a range of about 45 degrees (°) to about 180 degrees (°), such as about 60 degrees (°) to about 160 degrees (°), such as about 75 degrees (°) to about 145 degrees (°), such as about 140 degrees (°).

On the other hand, “field of illumination” or “FOI” refers to the area over which the LDAF sensor 400 provides illumination. More particularly, the FOI 550 of the example LDAF sensor 400 describes the coverage area illuminated by the optical signals (e.g., laser pulses) emitted by the emitter 402. The FOI 550 for the LDAF sensor 400 may be affected by a variety of factors, such as type and power of the emitter, hardware, optical design, distance between the emitter and target (e.g., subject), and the like. As shown in FIG. 7, the FOI 550 of the example LDAF sensor 400 corresponds to the FOV 500 of the example LDAF sensor 400 depicted in FIG. 6. Moreover, as further shown in FIG. 7, the FOI 550 of the example LDAF sensor 400 includes approximately zero percent illumination in an area 552 that corresponds to the collector exclusion zone 502 depicted in FIG. 6.

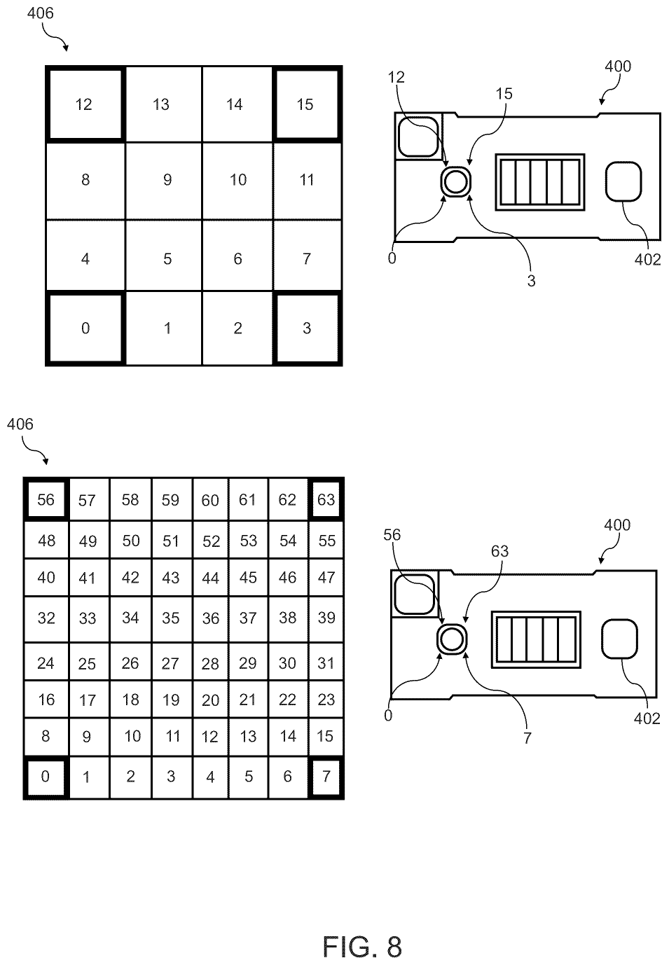

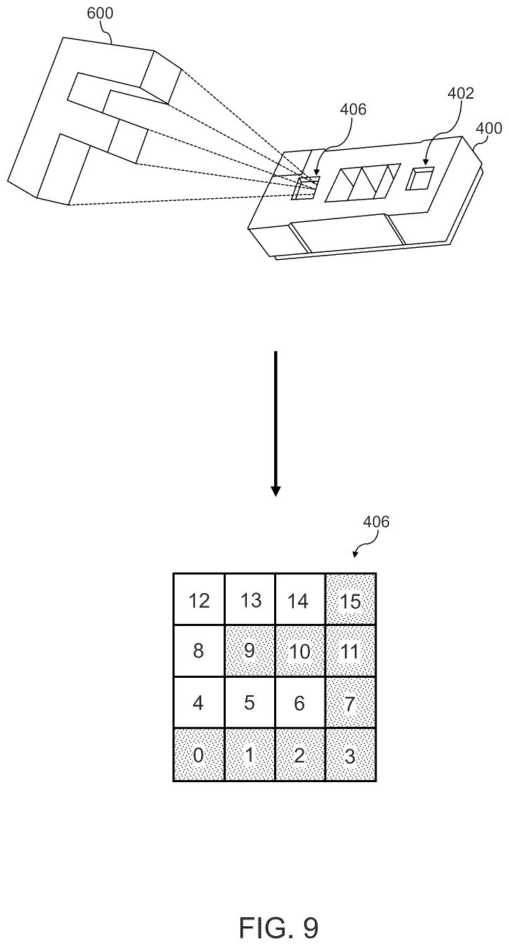

FIG. 8 depicts example zone mappings of the example LDAF sensor 400 according to example embodiments of the present disclosure. As noted above, the LDAF sensor 400 may include an array of collectors 404 arranged in a grid-like pattern. In some examples, the LDAF sensor 400 may include a collector array 406 having sixteen collectors 404 arranged in a four-by-four grid. In such examples, as shown in FIG. 9, the LDAF sensor 400 may have sixteen “zones” (e.g., zone 0, zone 1, zone 2, . . . , zone 15). More particularly, the LDAF sensor 400 may have four “corner zones” (e.g., zone 0, zone 3, zone 12, zone 15) and twelve “inner zones” (e.g., zones 1-2, zones 4-11, zones 13-14). Additionally and/or alternately, in some examples, the LDAF sensor 400 may include a collector array 406 having sixty-four collectors arranged in an eight-by-eight grid. In such examples, the LDAF sensor 400 may have sixty-four “zones” (e.g., zone 0, zone 1, zone 2, . . . , zone 63). More particularly, the LDAF sensor 400 may have four “corner zones” (e.g., zone 0, zone 7, zone 56, zone 63) and sixty “inner zones” (e.g., zones 1-6, zones 8-55, zones 57-62).

FIG. 9 depicts an example SPAD array (e.g., collector array 406) zone mapping of the example LDAF sensor 400 according to example embodiments of the present disclosure. As shown in the example depicted in FIG. 9, an emitter 402 of the LDAF sensor 400 may emit one or more optical signals (e.g., laser pulses) towards a target 600. In the example of FIG. 9, the target 600 is a shaped like an uppercase “F.” As shown, one or more of the optical signals (e.g., laser pulses) emitted by the emitter 402 may reflect off the target and, subsequently, may be detected by the collector array 406. Due to the optical design of the collector array 406, however, zone 0 (e.g., bottom left) of the collector array 406 is illuminated by the top-right side of the target 600. Hence, the zone mapping of the collector array 406 illuminated by the reflected optical signals (e.g., reflected laser pulses) may be inverted in relation to the target 600 itself. That is, zones 0-3, 7, 9-11, 15 of the collector array 406 may be illuminated by the reflected optical signals (e.g., reflected laser pulses) which, as shown in FIG. 9, corresponds to an inverted uppercase “F.”

It should be understood that the LDAF sensor 400 is depicted in FIG. 9 as having sixteen collectors 404 arranged in a four-by-four array for case of illustration and discussion. Those having ordinary skill in the art, using the disclosures provided herein, will appreciate that a collector array 406 having any suitable number of collectors 404 may be used without deviating from the scope of the present disclosure.

As described herein, example aspects of the present disclosure are directed to obtaining optical sensor data via an optical sensor (e.g., LDAF sensor 400) of the mobile computing device 100 (FIGS. 1-4). As will now be described, the mobile computing device 100 may be operable to process the optical sensor data to determine a proximity metric for the user and, subsequent to determining the proximity metric for the user, determine one or more biometrics for the user.

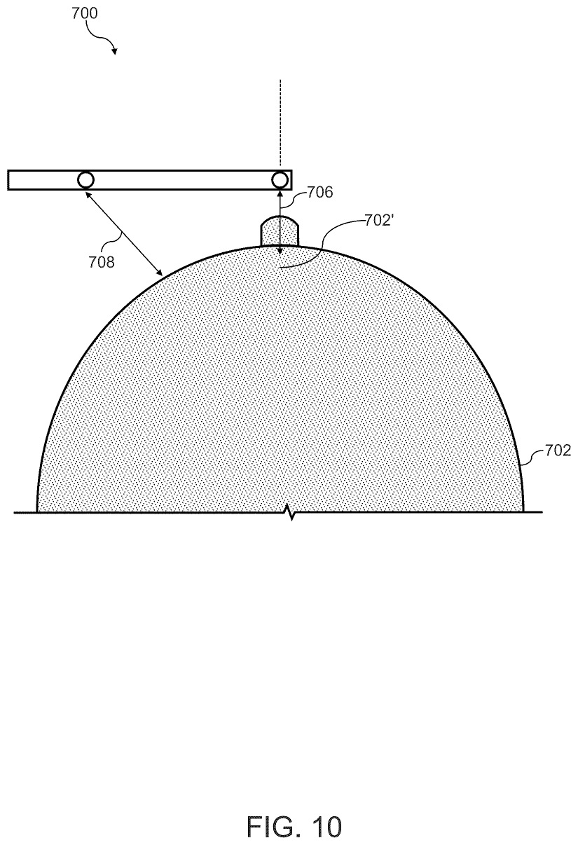

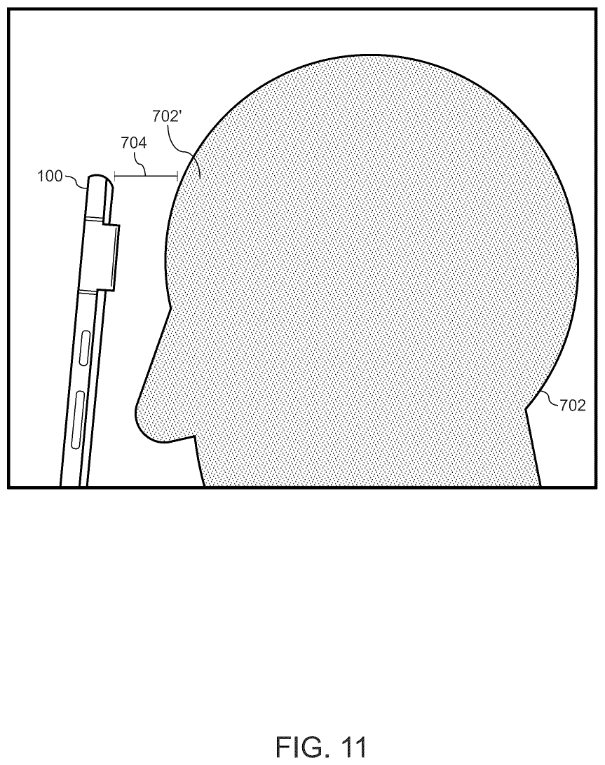

FIGS. 10-11 depict an example spatial arrangement 700 of the mobile computing device 100 with respect to a user 702 during the example proximity-determination operations described herein according to example embodiments of the present disclosure. FIGS. 10-11 will be discussed in conjunction with FIGS. 1-9.

More particularly, as shown, the mobile computing device 100 may be separated from the user 702 by a separation distance 704. In some examples, such as that shown in FIGS. 10-11, the separation distance 704 may correspond to a distance 706 between a forehead 702′ of the user 702 and the LDAF sensor 400 of the mobile computing device 100. Additionally and/or alternatively, in some examples, the separation distance 704 may correspond to a distance 708 between the forehead 702′ of the user 702 and the biometric sensor(s) 160 (e.g., temperature sensor(s)) of the mobile computing device 100.

As described herein, the user 702 must be within a threshold testing distance from the mobile computing device 100 to ensure accurate biometric readings (e.g., via the biometric sensor(s) 160). By way of non-limiting example, the threshold testing distance may be a distance less than and/or equal to 50 millimeters, such as a distance in a range of about 2 millimeters to about 50 millimeters, such as a range of about 5 millimeters to about 40 millimeters, such as a range of about 7.5 millimeters to about 30 millimeters, such as a range of about 10 millimeters to about 20 millimeters, such as a range of about 12.5 millimeters to about 17.5 millimeters, such as a range of about 14 millimeters to about 16 millimeters.

Due to various environmental factors, the LDAF sensor 400 may not provide precise measurements below the threshold testing distance. However, example aspects of the present disclosure provide for accurate temperature measurements at any distance below the threshold testing distance. For instance, as an illustrative example, the user 702 may be ten millimeters from the mobile computing device 100, but the LDAF sensor 400 may determine that the user 702 is approximately five millimeters from the mobile computing device 100. In such instances, however, the mobile computing device 100 may nevertheless provide an accurate temperature biometric reading, because the user 702 is within the threshold testing distance from the mobile computing device 100. Conversely, as another illustrative example, the user 702 may be approximately twenty millimeters from the mobile computing device 100. In such instances, if the LDAF sensor 400 determines that the user 702 is within the threshold testing distance from the mobile computing device 100, the mobile computing device 100 may provide an inaccurate temperature biometric reading due to the user 702 being outside of the threshold testing distance.

Accordingly, example aspects of the present disclosure are directed to a mobile computing device 100 that is operable to implement ranging algorithm 800 (e.g., similar to the ranging algorithm 166 (FIGS. 1-4)), thereby ensuring the user 702 is within the threshold distance from the mobile computing device 100 for accurate temperature biometric readings.

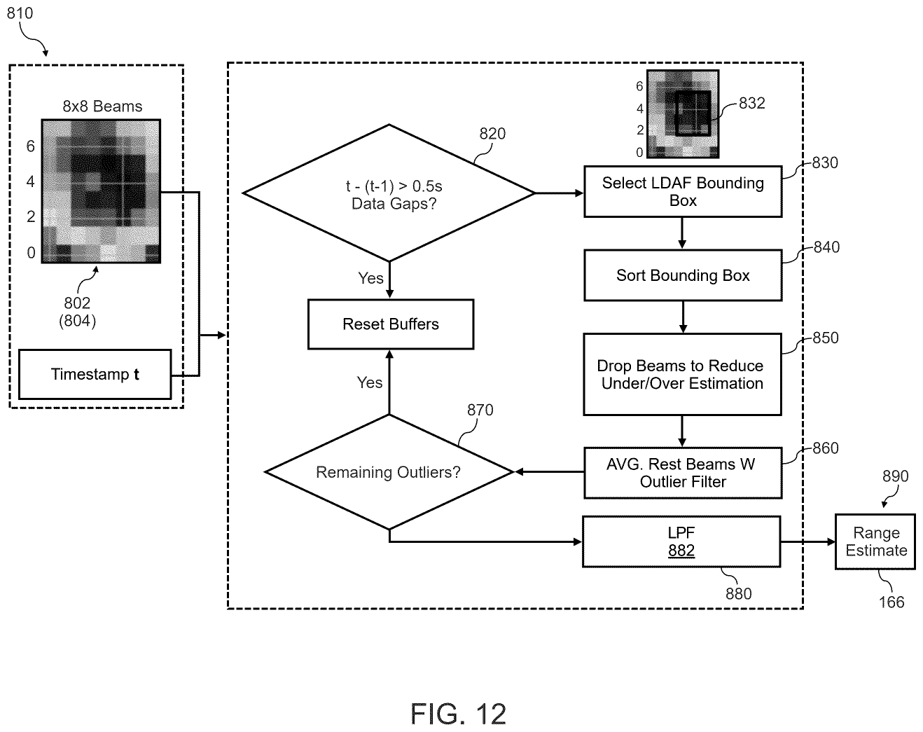

FIG. 12 depicts a flow chart diagram of an example ranging algorithm 800 according to example embodiments of the present disclosure. FIG. 12 will be discussed in conjunction with FIGS. 1-11.

As shown at (810), the mobile computing device 100 may obtain optical sensor data via an optical sensor, such as the LDAF sensor 400 described above. It should be understood that the optical sensor data includes a plurality of measurement frames, such as the example measurement frame 802. As shown, each measurement frame 802 includes a plurality of reflected optical signals 804 respectively associated with a plurality of optical signals emitted by the optical sensor (e.g., LDAF sensor 400). For instance, in the example of FIG. 12, each measurement frame 802 includes sixty-four individual reflected optical signals 804 corresponding that each correspond to one of a plurality of optical signals emitted by the LDAF sensor 400.

At (820), the mobile computing device 100 may determine a temporal relationship between the measurement frame 802 and a preceding measurement frame in order to determine whether the measurement frame 802 is temporally correlated with the preceding measurement frame.

At (830), in response to determining that the measurement frame 802 is temporally correlated with the preceding measurement frame, the mobile computing device 100 may determine a measurement region-of-interest (ROI) 832 for the measurement frame 802. As shown, the measurement ROI 832 may include a first subset of the reflected optical signals 804 of the measurement frame 802. In some examples, the mobile computing device 100 may store the measurement ROI 832 in a memory, such as the memory 154 (FIGS. 1-4).

At (840), the mobile computing device 100 may sort the first subset of reflected optical signals 804 in the measurement ROI 832 based on a magnitude of the respective reflected optical signals 804 in the measurement ROI 832.

At (850), the mobile computing device 100 may identity and discard one or more of the reflected optical signals 804 in the first subset (e.g., measurement ROI 832) to generate a second subset of reflected optical signals 804. More particularly, in some examples, the mobile computing device 100 may determine that one or more of the plurality of reflected optical signals 804 in the measurement ROI 832 are invalid optical signals based, for instance, on the respective magnitudes of each reflected optical signal 804. For instance, in some examples, an invalid optical signal may correspond to a reflected optical signal 804 in the measurement ROI 832 having a smallest magnitude relative to the other reflected optical signals 804 in the measurement ROI 832. In some examples, an invalid optical signal may also correspond to a reflected optical signal 804 in the measurement ROI 832 having the greatest magnitude relative to the other reflected optical signals 804 in the measurement ROI 832.

In some examples, the one or more invalid optical signals may be a plurality of invalid optical signals. For instance, in some examples, the plurality of invalid optical signals may correspond to the two reflected optical signals 804 having the two smallest magnitudes, the three reflected optical signals 804 having the three smallest magnitudes, the four reflected optical signals 804 having the four smallest magnitudes, the five reflected optical signals 804 having the five smallest magnitudes, the six reflected optical signals 804 having the six smallest magnitudes, the seven reflected optical signals 804 having the seven smallest magnitudes, the eight reflected optical signals 804 having the eight smallest magnitudes, etc., relative to the other reflected optical signals 804 in the measurement ROI 832. Additionally and/or alternatively, in some examples, the plurality of invalid optical signals may correspond to the two reflected optical signals 804 having the two greatest magnitudes, the three reflected optical signals 804 having the three greatest magnitudes, the four reflected optical signals 804 having the four greatest magnitudes, the five reflected optical signals 804 having the five greatest magnitudes, the six reflected optical signals 804 having the six greatest magnitudes, the seven reflected optical signals 804 having the seven greatest magnitudes, the eight reflected optical signals 804 having the eight greatest magnitudes, etc., relative to the other reflected optical signals 804 in the measurement ROI 832. Those having ordinary skill in the art, using the disclosures provided herein, will understand that any suitable number of reflected optical signals 804 may be identified as invalid optical signals and subsequently discarded to generate the second subset of reflected optical signals 804 without deviating from the scope of the present disclosure.

In some examples, the mobile computing device 100 may determine that a magnitude of one or more of the reflected optical signals 804 in the measurement ROI 832 is indicative of collector saturation (e.g., saturation of the collector array 406) and, in response, may determine that the particular reflected optical signal 804 is an invalid optical signal. Additionally and/or alternatively, in some examples, the mobile computing device 100 may determine that a corresponding collector 404 (FIGS. 7-11) that received a particular reflected optical signal 804 is within a threshold distance to the emitter 402 (FIGS. 7-11) of the LDAF sensor 400 and, in response, may determine that the particular reflected optical signal 804 is an invalid optical signal. Once the invalid optical signals are identified, the mobile computing device 100 may discard the invalid optical signals to generate the second subset of the reflected optical signals 804, which includes only the remaining valid reflected optical signals.

At (860), the mobile computing device 100 may determine an average magnitude of the second subset of reflected optical signals 804 (e.g., generated at (840)).

At (870), the mobile computing device 100 may perform an outlier check based on the respective magnitudes of the reflected optical signals 804 in the second subset (e.g., generated at (840)).

At (880), the mobile computing device 100 may provide the average magnitude (e.g., determined at (860)) to a low-pass filter (LPF) 882, which is configured to maintain an average magnitude of a plurality of measurement frames 802, such as a range of about five measurement frames 802 to about ten measurement frames 802.

The processing steps (810)-(880) may be performed for each measurement frame 802 of reflected optical signals 804 obtained by the mobile computing device 100. Hence, after a threshold number of measurement frames 802 are obtained and processed via the ranging algorithm 800, the mobile computing device 100 may, at (890), determine the proximity metric 168 for the user of the mobile computing device 100 based on the average magnitude of the plurality of measurement frames 802 as determined by the LPF 882 (e.g., at (880)).

Subsequent to determining the proximity metric 168 at (890), the mobile computing device 100 may be configured to determine a biometric for the user, such as a body temperature of the user. More particularly, as an illustrative example, the mobile computing device 100 may determine whether the user is within a threshold testing distance (e.g., less than about 30 millimeters) from the mobile computing device 100 based on the proximity metric 168 determined at (890). In examples where the user is within the threshold testing distance from the mobile computing device 100, the mobile computing device 100 may generate a notification for display to the user via one of the output devices 146 (FIGS. 1-4) that indicates the user is within the threshold testing distance. The notification may be any suitable notification, such as an auditory notification, a visual notification, a haptic notification, and/or the like. The mobile computing device 100 may then obtain biometric sensor data (e.g., temperature data) via the biometric sensor(s) 160 (e.g., via temperature sensors) and may determine one or more biometrics associated with the user based on the biometric data (e.g., a body temperature of the user). Hence, by ensuring the user is within the threshold testing distance, the mobile computing device 100 is able to ensure subsequent biometric readings made by the mobile computing device 100 are accurate.

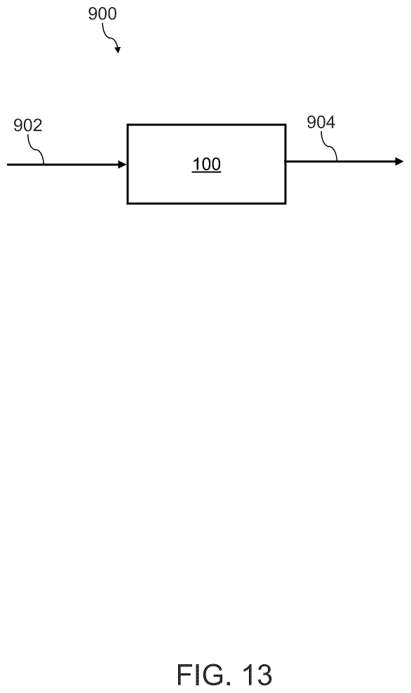

To further ensure the subsequent biometric readings made by the mobile computing device 100 are accurate, the mobile computing device 100 may be further configured to implement a skin-gating check to ensure the optical signals emitted by the LDAF sensor 400 are directed to human skin (e.g., in a direction towards the user of the mobile computing device 100). For instance, FIG. 13 depicts a block diagram of an example skin-gating framework 900 according to example embodiments of the present disclosure. The skin-gating framework 900 may be implemented concurrently to and/or consecutively with the ranging algorithm 800 described above (FIG. 12). As shown, the mobile computing device 100 may obtain temperature sensor data 902 associated with an object in the FOV 500 of the LDAF sensor 400 and may determine a temperature 904 of the object in the FOV 500 of the LDAF sensor 400 based on the temperature sensor data 902. If the temperature 904 of the object is within a threshold temperature range, such as a range of about 30 degrees C. to about 40 degrees C., the mobile computing device 100 may determine that the user is within the FOV 500 of the LDAF sensor 400. In this way, the mobile computing device 100 is operable to ensure that the optical signals emitted by the LDAF sensor 400 are directed to human skin (e.g., in a direction towards the user of the mobile computing device 100).

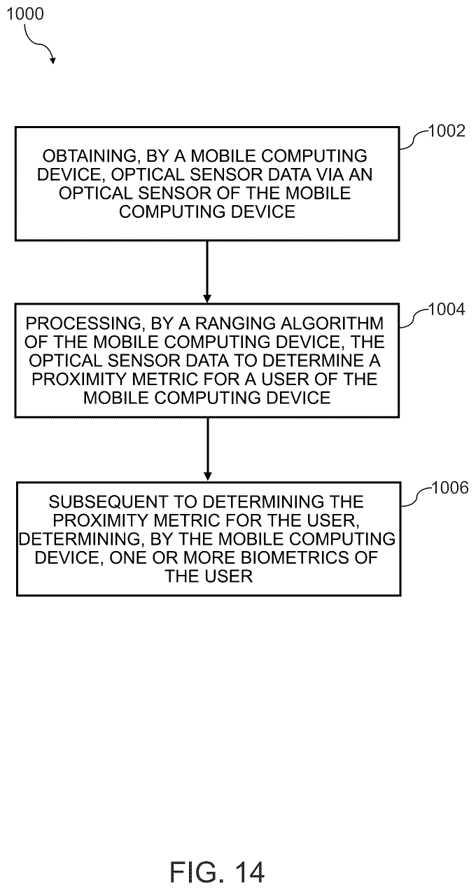

FIG. 14 depicts a flow chart diagram of an example method 1000 according to example embodiments of the present disclosure. In some examples, the method 1000 may be implemented using, for instance, the mobile computing device 100 described herein. Additionally and/or alternatively, in other examples, the method 1000 may be implemented by a computing device (e.g., server, smartphone, etc.) that is communicatively coupled to the mobile computing device 100. It should be understood that, in some examples, some steps of the method 1000 may be implemented locally on the mobile computing device 100, whereas other steps of the method 1000 may be implemented by a computing device that is remote from the mobile computing device 100 and is communicatively coupled to the mobile computing device 100 via one or more wireless networks (e.g., network 300).

FIG. 14 depicts steps performed in a particular order for purposes of illustration and discussion. Those having ordinary skill in the art, using the disclosures provided herein, will understand that various steps of any of the methods described herein may be omitted, expanded, performed simultaneously, rearranged, and/or modified in various ways without deviating from the scope of the present disclosure. Furthermore, various steps (not illustrated) may be performed without deviating from the scope of the present disclosure. Additionally, although the method 1000 is generally discussed with reference to the mobile computing device 100 described herein, those having ordinary skill in the art, using the disclosures provided herein, will understand that aspects of the present method 1000 may find application with any suitable computing device.

Referring now to FIG. 14 at (1002), the method 1000 may include obtaining, by a mobile computing device, optical sensor data via an optical sensor of the mobile computing device. At (1004), the method 1000 may include processing, by a ranging algorithm of the mobile computing device, the optical sensor data to determine a proximity metric for a user of the mobile computing device. At (1006), subsequent to determining the proximity metric for the user (e.g., at (1004), the method 1000 may include determining, by the mobile computing device, one or more biometrics of the user.

While the present subject matter has been described in detail with respect to specific example embodiments thereof, it will be appreciated that those skilled in the art, upon attaining an understanding of the foregoing can readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A method, comprising:
obtaining, by a mobile computing device, optical sensor data via an optical sensor of the mobile computing device;
processing, by a ranging algorithm of the mobile computing device, the optical sensor data to determine a proximity metric for a user of the mobile computing device; and
subsequent to determining the proximity metric for the user, determining, by the mobile computing device, one or more biometrics of the user.

2. The method of claim 1, wherein obtaining the optical sensor data comprises:
determining, by the mobile computing device, that the user is within a field-of-view (FOV) of a laser direct autofocus (LDAF) sensor of the mobile computing device;
emitting, by an emitter of the LDAF sensor, one or more optical signals in a direction towards the user; and
receiving, by a collector array of the LDAF sensor, one or more reflected optical signals associated with the one or more optical signals emitted by emitter, the collector array comprising a plurality of collectors.

3. The method of claim 2, wherein the collector array comprises at least sixteen collectors.

4. The method of claim 2, wherein determining that the user is within the FOV of the LDAF sensor comprises:
obtaining, by a temperature sensor of the mobile computing device, temperature sensor data associated with an object in the FOV of the LDAF sensor;
determining, by the mobile computing device, a temperature of the object in the FOV of the LDAF based on the temperature sensor data;
determining, by the mobile computing device, that the temperature of the object in the FOV of the LDAF is within a threshold temperature range; and
in response to determining that the temperature of the object in the FOV of the LDAF sensor is within the threshold temperature range, determining, by the mobile computing device, that the user is within the FOV of the LDAF sensor.

5. The method of claim 4, wherein the threshold temperature range comprises temperatures in a range of about 30 degrees C. to about 40 degrees C.

6. The method of claim 1, wherein the optical sensor data comprises a plurality of measurement frames, each measurement frame comprising a plurality of reflected optical signals respectively associated with a plurality of optical signals emitted by the optical sensor, and wherein processing the optical sensor data to determine the proximity metric comprises:
for each measurement frame:
determining, by the mobile computing device, a temporal relationship between the measurement frame and a preceding measurement frame of the plurality of measurement frames;
determining, by the mobile computing device, that the measurement frame is temporally correlated with the preceding measurement frame based on the temporal relationship;
in response to determining that the measurement frame is temporally correlated with the preceding measurement frame, determining, by the mobile computing device, a measurement region-of-interest (ROI) for the measurement frame, the measurement ROI comprising a subset of the plurality of reflected optical signals of the measurement frame; and
storing, by the mobile computing device, the measurement ROI for each measurement frame in a memory of the mobile computing device.

7. The method of claim 6, wherein the subset is a first subset, and wherein processing the optical sensor data to determine the proximity metric further comprises:
for each measurement frame:
sorting, by the mobile computing device, the first subset of the plurality of reflected optical signals of the measurement ROI based on a magnitude of the respective reflected optical signal of the first subset;
determining, by the mobile computing device, that one or more of the plurality of reflected optical signals in the measurement ROI are invalid optical signals based on the respective magnitude;
discarding, by the mobile computing device, the invalid optical signals from the subset to generate a second subset comprising a plurality of valid reflected optical signals; and
determining, by the mobile computing device, an average magnitude of the second subset based on the respective magnitude of each valid reflected optical signal of the plurality of valid reflected optical signals; and
determining, by the mobile computing device, the proximity metric for the user based on the average magnitude of the second subset of each of the plurality of measurement frames.

8. The method of claim 7, wherein determining that one or more of the plurality of reflected optical signals in the measurement ROI are invalid optical signals comprises:
for at least one reflected optical signal in the measurement ROI:
determining, by the mobile computing device, that a magnitude of the reflected optical signal is indicative of collector saturation; and
in response to determining that the magnitude of the reflected optical signal is indicative of collector saturation, determining, by the mobile computing device, that the reflected optical signal is an invalid optical signal.

9. The method of claim 7, wherein determining that one or more of the plurality of reflected optical signals in the measurement ROI are invalid optical signals comprises:
for at least one reflected optical signal in the measurement ROI:
determining, by the mobile computing device, that a collector of the optical sensor that received the reflected optical signal is within a threshold distance to an emitter of the optical sensor; and
in response to determining that the collector of the optical sensor that received the reflected optical signal is within the threshold distance to the emitter, determining, by the mobile computing device, that the reflected optical signal is an invalid optical signal.

10. The method of claim 7, wherein the invalid optical signals comprise:
a reflected optical signal having a smallest magnitude of the plurality of reflected optical signals in the measurement ROI; and
a reflected optical signal having a greatest magnitude of the plurality of reflected optical signals in the measurement ROI.

11. The method of claim 1, wherein determining the one or more biometrics of the user comprises:
determining, by the mobile computing device, that the user is within a threshold testing distance from the mobile computing device based on the proximity metric;
in response to determining that the user is within the threshold testing distance from the mobile computing device, generating, by the mobile computing device, a notification for display to the user via an output device of the mobile computing device, the notification indicating to the user that the user is within the threshold testing distance from the mobile computing device;
obtaining, by the mobile computing device, biometric sensor data via one or more biometric sensors of the mobile computing device; and
determining, by the mobile computing device, the one or more biometrics of the user based on the biometric sensor data.

12. The method of claim 11, wherein the threshold testing distance is less than about 30 millimeters.

13. The method of claim 11, wherein the notification is one of:
an auditory notification;
a visual notification; or
a haptic notification.

14. The method of claim 1, wherein the one or more biometrics of the user comprises a body temperature of the user.

15. The method of claim 1, wherein the optical sensor is a laser direct autofocus (LDAF) sensor.

16. The method of claim 15, wherein the LDAF sensor comprises a sampling rate in a range of about 5 Hz to about 30 Hz.

17. The method of claim 1, wherein the proximity metric corresponds to a separation distance between the mobile computing device and a body part of the user.

18. The method of claim 17, wherein the separation distance is a distance between a forehead of the user and the optical sensor of the mobile computing device.

19. A mobile computing device, comprising:
a display;
an image capture assembly comprising an imaging device;
an optical sensor;
a biometric sensor; and
one or more processors configured to:
obtain optical sensor data;
process, with a ranging algorithm, the optical sensor data to determine a proximity metric for a user of the mobile computing device; and
subsequent to determining the proximity metric for the user, determine one or more biometrics of the user.

20. One or more non-transitory computer-readable media collectively storing instructions that, when executed by one or more processors of a mobile computing device, cause the mobile computing device to perform operations, the operations comprising:
obtaining optical sensor data via an optical sensor of the mobile computing device;
processing, by a ranging algorithm, the optical sensor data to determine a proximity metric for a user of the mobile computing device; and
subsequent to determining the proximity metric for the user, determining one or more biometrics of the user.
